(19) United States
(12) Patent Application Publication
(10) Pub. No.: US 2021/0100507 A1
(43) Pub. Date: Apr. 8, 2021

(54) Device for Detecting at Least One Human Vital Parameter by Means of a Sensor

(71) Applicant: Neuroloop GmbH, Freiburg (DE)

(72) Inventors: Fabian KIMMIG, Freiburg (DE); Tim BORETIUS, Freiburg (DE); Dennis PLACHTA, Voerstetten (DE)

(21) Appl. No.: 16/620,667

(22) PCT Filed: May 28, 2018
(86) PCT No.: PCT/EP2018/063863

§ 371 Date: Dec. 9, 2019

(30) Foreign Application Priority Data
Jun. 9, 2017 (DE) 10 2017 209 767.1

(51) Int. Cl. A61B 5/00; A61B 5/0205

(52) U.S. Cl. CPC A61B 5/6817; A61B 5/6843; A61B 5/02438; A61B 2562/0219; A61B 5/02055

(57) ABSTRACT

A device for the sensory detection of at least one human vital parameter comprises a support, which is shaped and dimensioned to be at least partly removably placed in the human outer ear canal, and to which at least one sensor for the detection of a vital parameter is attached. The support is a planar substrate, which is a hollow cylinder defining a continuously open hollow channel. A hollow cylindrical outer wall region at least partly contacts a surface of the inner wall of the ear canal when the support is at least partly placed in the human outer ear canal.

### CROSS REFERENCE TO RELATED APPLICATION

Reference is made to PCT/EP2018/063863 filed May 28, 2018, designating the United States, which claims priority to German Application No. 10 2017 209 767.1 filed Jun. 9, 2017, which are incorporated herein by reference in their entirety

### BACKGROUND OF THE INVENTION

### Field of the Invention

The invention relates to a device for detecting at least one human vital parameter by a sensor, comprising a support, which is suitably shaped and dimensioned to be removably placed at least partly in the human outer ear canal, and to which at least one sensor is attached in order to detect a vital parameter.

### Description of the Prior Art

Generic devices are small lightweight units, which are designed for attachment to the ear for sensory vital parameter recording, under the condition that, on the one hand, they do not present any restrictions or hindrances for the person, and, on the other hand, they are almost invisible, or invisible, in appearance to third parties. Sensor systems of known art that are to be attached on or in the ear are able to record vital parameters, such as blood pressure, blood oxygen saturation, ECG signal, heart rate, etc. by a sensor, and transmit them wirelessly, or in a wired manner, to a data recording and evaluation unit for further data evaluation.

A robust optoelectronic cardiovascular monitoring device, which can be positioned in the ear, and which, for purposes of secure attachment to the ear, has an ergonomically adapted hook section, which is to be arranged behind the auricle, and which is connected to a housing part, which opens out at least partly into the outer ear canal, and thus closes the ear canal mechanically as well as acoustically, can be found in the publication EP 2 116 138 B1. In addition to a motion sensor integrated into the hook section, at least one optical emitter is provided which emits light that radiates onto the rear of the auricle. The light components transmitted through the auricle are detected by an optical receiver attached to the housing part, and are then subjected to photoplethysmographic evaluation, which takes place in an external evaluation unit, to which the detected light signals are transmitted, either in a wired manner or wirelessly. Optionally, additional signal and receiving electrodes for the acquisition of electrocardiological signals can be provided on the hook section, and/or on the housing part.

Although the sensor system of known art that is to be attached to the ear largely meets the requirements with regard to wearing comfort, freedom of movement and optical inconspicuousness, the plug-like housing part, which opens out into the outer ear canal, closes the ear canal in a soundproof and airtight manner, as a result of which the acoustic perception of the ear concerned is at least severely impaired.

U.S. Pat. No. 6,454,718 discloses a cylindrical sensor support, which is inserted into the outer ear canal, and has a large number of sensors, for example a strain sensor for blood pressure measurement, an oxygen sensor, etc.

The publication EP 1 594 340 A2 describes a funnel-shaped holder for insertion into the human outer ear canal, into which an acoustic transmission channel can be connected, for connection to a hearing aid.

In each of the two cases described above, there is no free access to the outer ear canal. The outer ear canal is in each case closed acoustically by the two devices of known art.

In US published application 2003/0233051 A1, which describes a variant of the above monitoring device, the circumstances of acoustic decoupling as a result of the housing part protruding into the outer ear canal are used for this purpose, and/or are thereby avoided, by integrating a loudspeaker into the housing part, which is connected, for example, to an acoustic signal source, for example in the form of an MP3 player, or a microphone, for the purpose of transmitting environmental sounds.

### SUMMARY OF THE INVENTION

The invention is based on developing a device for the sensory detection of at least one human vital parameter with a support, which is suitably shaped and dimensioned to be at least partly removably placed in the human outer ear canal, and to which at least one sensor for the detection of a vital parameter is attached so that the wearing comfort is further improved. In particular, the outer ear canal must not be closed acoustically, so that any systems for transmitting or amplifying environmental sounds can be eliminated. In the same manner, the optical inconspicuousness must be maintained or improved.

In accordance with the invention, a device for the sensory detection of at least one human vital parameter is designed such that the support, on or in which at least one sensor for the detection of a vital parameter is mounted, is constructed as a planar substrate, which is shaped in the form of a hollow cylinder, with outwardly delimits a continuously open hollow channel radially to a cylindrical axis paired with the hollow cylinder, and has a hollow cylindrical outer wall region that at least partly contacts the surface of the inner wall of the ear canal when the support is at least partly placed in the human outer ear canal.

The shape of the support in the form of a thin-walled hollow cylinder, which in its inserted state nestles closely against the inner wall of the ear canal, means that the free access to the outer ear canal remains essentially unaffected. In this manner, the acoustic perception of the person concerned is not affected for the duration of the insertion of the hollow cylindrical support into the outer ear canal. Also, the natural stimulus for pressure compensation remains unchanged in the event of abrupt pressure changes.

In a preferred embodiment, the support is a skin-friendly planar substrate, preferably of a single-layer or multi-layer, film polymer material, which is convertible into the shape of a hollow cylinder by winding around a winding axis. The film planar substrate typically has a substrate thickness of 10 μm to a few 100 μm, and, when wound, encloses a hollow cylinder whose hollow cylindrical inner diameter is negligibly smaller than the diameter of the human outer ear canal, which is typically between 4 and 7 mm. Even in the case of a multiple winding of the planar substrate around the winding axis and the multi-layer film overlap associated with this action, the hollow cylinder wall thickness thereby formed is negligible with respect to the dimensions of the human ear canal, so that no, or only negligibly minor, acoustic restrictions are associated with the use of the device of the invention in the outer ear canal, and, furthermore, a largely unaltered ventilation of the outer ear canal is ensured.

The planar substrate is preferably square or rectangular in the non-wound state and has an upper and a lower surface. Other planar substrate forms are also conceivable, as explained in the context of the drawings. Depending on the particular measurement requirements profile, a certain number and different types of sensors are to be integrated into or applied onto the film-like planar substrate, whose task is to record physiological vital parameters of a person.

The thin-film technology known per se is preferably suited for this purpose, with which it is possible to integrate microsystem sensors into, or apply them onto, the planar substrate. The flexibility and the winding capability of the planar substrate, which is preferably made of a multi-layer, film polymer material, is not at all, or is only insignificantly, impaired by the sensors, so that a subsequent winding of the planar substrate, populated and fitted with the vital sensors, into a hollow cylinder is possible.

The winding process preferably takes place parallel to a side edge of the square or rectangular planar substrate, such that the planar substrate formed by winding has two opposing planar substrate sections in the form of a hollow cylinder, which overlap each other loosely as a result of the winding process, and without external mechanical force. The degree of mutual overlap can basically be selected in any manner. As already mentioned, it is advantageous for reasons of the least possible impairment of the outer ear canal to transform the planar substrate into a winding, by forming a single-layer hollow cylinder wall, so that the opposing planar substrate sections overlap only slightly from the winding.

At the same time it is possible to transform the planar substrate into a multi-layer hollow cylinder wall by multiple spiral windings around a winding axis.

In order to prevent the sheet of a planar substrate, which preferably is polyimide, and serves as a support for the sensors, from autonomously moving back into its initial shape after winding. The planar substrate is wound into a hollow cylinder which is subjected to a heat treatment. As a result, the planar substrate obtains a shape-retaining stiffness inherent in the material and retains its hollow cylinder shape with long-term stability.

As an alternative to forming the planar substrate by a winding process, it is also possible to form the hollow cylinder shape by a casting process, in which the planar substrate is produced in a configuration wound up into a hollow cylinder.

It is also possible to produce the support, by a mould casting process, in the form of a closed hollow cylinder from a compressible material, e.g. from a polymer foam. In this case, the hollow cylindrical shape of the support without external mechanical constraint has a first outer cylinder diameter which is greater than a second outer cylinder diameter that the support occupies when placed at least partly within the human outer ear canal, wherein the support exerts a force that is directed radially outwards onto the outer ear canal by the restoring forces inherent to the material, and which inserts the support securely and firmly within the outer ear canal.

In all the possible forms of embodiment mentioned above, the planar substrate which is formed into a hollow cylinder comprises a hollow cylindrical passageway, through which the outer ear canal remains ventilated, and is thus fully acoustically coupled to the environment. In addition, natural pressure equalization and forced pressure equalisation remain possible in this manner.

For purposes of vital parameter acquisition, a number of different sensors are available for integration into, or application onto, the planar substrate. Thus, for example, the heart rate can be implemented with the aid of a capacitive sensor element, for example designed as an interdigital electrode structure integrated within the planar substrate. If the body temperature is to be measured, temperature sensors based on electrical resistance changes, for example PT 100, are suitable for this purpose. For the detection of blood oxygen saturation, suitably selected illuminants and appropriately selected photodetectors are suitable, which for the purposes of light emission as well as detection are arranged on an upper side of the support or planar substrate facing the outer ear canal, and are able to generate measurement signals for evaluation on the basis of photoplethysmography.

Electrode contacts attached to the surface of the planar substrate facing the outer ear canal are also suitable for picking up electrocardiographic signals via skin contact with the inner ear wall, to generate an electrocardiogram (ECG). Further sensors, such as acceleration sensors, etc., can also be integrated in the planar substrate.

In a further embodiment, at least one actuator is mounted on the support, preferably in the form of a sound-generating actuator. In this manner some forms of tinnitus can be pushed back into the psychoacoustic background by the acoustic superimposition of an artificially generated noise with a particular noise spectrum. Such noise can be implemented with the aid of a “miniature loudspeaker” that is integrated into the planar substrate, preferably with a sound radiation surface facing the inner wall of the hollow cylinder. Such an actuator integrated in the planar substrate would have the merit that external sounds could reach the ear unhindered, while nevertheless the actuator would be “working” continuously against the tinnitus. Such actuators or sound transducers are preferably in the form of small ceramic elements, e.g. lead zirconate titanate.

All sensors integrated into, or applied onto, the support, as well as any actuators, are preferably connected in a wired manner to the electronic module, which contains an electrical energy source as well as the evaluation electronics required for signal processing and evaluation. The electronic module can preferably be fixed inconspicuously behind the auricle with the aid of a mounting, ergonomically adapted to the ear, as a separate unit from the support.

Alternatively or in combination, it is possible to integrate a receiver and transmitter unit suitable for energy and signal transmission into the hollow cylindrical support, e.g. on the basis of RFID technology, so that it is possible to operate all sensors placed in or on the support wirelessly. This requires an additional microelectronic unit integrated in the support, to which electrical energy and control signals from an external control unit are transmitted for the supply and control of the sensors, and via which the sensory vital parameters recorded are transmitted in the form of sensor signals to the control unit for further evaluation. By using such a wireless signal transmission technology, all that is required is a sensor support with a hollow cylindrical design, which is reliably fixed within the outer ear canal and is otherwise not visually perceptible to third parties.

In a particularly preferred form of embodiment, the function of the external control unit can be undertaken by a commercially available smart phone carried by the person, which already has all the hardware components required for wireless power and signal transmission to the microelectronic unit integrated on the support. The technical ability of the smart phone to communicate with the hollow cylindrical sensor support placed in the outer ear canal for the purpose of recording and transmitting vital parameters can be achieved by installing a program in the form of an app that has been designed for this purpose.

The device of the invention is particularly suitable for recording the following human vital parameters: heart rate, ECG signal, body temperature, blood oxygen saturation, CO₂ blood saturation, blood sugar, pH, skin resistance, and blood pressure.

### BRIEF DESCRIPTION OF THE DRAWINGS

The invention will be described below in an exemplary manner by way of examples of embodiments with reference to the figures, being without any limitation of the general inventive concept. Here:

FIG. 1a shows a schematic illustration of a flat planar substrate with vital parameter sensors;

FIG. 1b shows illustrations of the wound planar substrate in the form of a hollow cylinder,

FIG. 2 shows a schematic illustration of the hollow cylindrical sensor support placed in the outer ear canal,

FIG. 3 shows an integral embodiment of the cylindrical sensor support as a hollow cylinder, and

FIGS. 4a, b, and c show sensor supports with structured planar substrates.

### DETAILED DESCRIPTION OF THE INVENTION

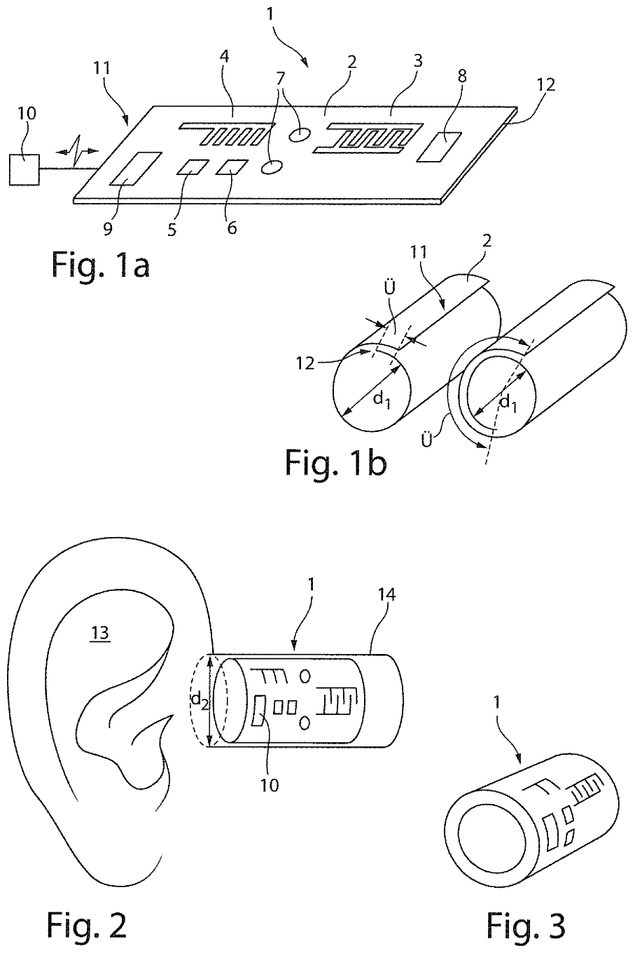

FIG. 1a shows in perspective a top view of a schematically depicted support 1 in the form of a planar, rectangular planar substrate 2 in the form of a single-layer or multi-layer polymer layer, which typically has a layer thickness of a few 10 μm to a few 100 μm. The substrate 1 is a single layer or multi-layer polymer layer. Different vital parameter sensors are integrated into/applied onto the planar substrate 2. The following vital parameter sensors 3, . . . , 8 can be mounted in any number and arrangement on or in the planar substrate 2. It is also possible to provide just a single vital parameter sensor on or in the planar substrate 2.

Suitable vital parameter sensors are: a capacitive interdigital structure 3 for recording heart rate, a resistance-based temperature sensor 4 which is preferably PT100 or PT1000 temperature sensor, for recording body temperature; an LED photodiode 5 together with a photodetector 6 for measuring blood oxygen saturation or CO₂ blood saturation based on photoplethysmography, ECG sensors 7 in the form of contact electrodes attached to the surface of the planar substrate, an acceleration sensor 8, and other appropriate microelectronic sensors, all of which are preferably applied onto or integrated into the planar substrate 2 using thin-film technology. All vital parameter sensors present on the planar substrate 2 are electrically connected to a microelectronic unit 9 via electrical conducting tracks (not shown), which supply the vital parameter sensors with both energy and control signals, and via which the recorded sensor signals are fed to the microelectronic unit 9 for further processing. In one example of embodiment, the microelectronics unit 9 is connected by cable to an external control unit 10, which serves to provide both the power and signal supplies.

In a particularly preferred form of embodiment, the signal and power transfers between the control unit 10 and the microelectronics 9 take place on the basis of RFID technology, or similar wireless power and signal transfer technology, so that the control unit 10 can be handled as a mobile unit separate from the support 1. Alternatively, a wired solution is also available.

FIG. 1b shows the planar substrate 2 in a form wound into a hollow cylinder, in which the opposing side edge regions 11, 12 of the planar substrate 2 slightly overlap. See the overlap U in the left-hand illustration in FIG. 1b. Alternatively, it is possible to form the winding of the planar substrate 2 with a large-area mutual overlap U of the opposing side edge regions 11, 12, as illustrated on the right-hand side in FIG. 1b.

After winding the film planar polymer substrate 2, in accordance with the hollow cylinder shapes shown in FIG. 1b, the wound planar substrate 2 is treated in such a way that the planar substrate 2 experiences a gain in shape-retaining stiffness inherent to the material, by which the hollow cylinder shape remains stable in the long term, for example by an annealing process.

The planar substrate 2 wound into a hollow cylinder has a cylinder diameter d1, which is slightly greater than the inner diameter d2 of the outer ear canal G of a person. See also FIG. 2 which schematises a human auricle 13 with an outer ear canal 14, into which a sensor support 1 is inserted. By virtue of the smaller inner diameter d2 the hollow cylindrical sensor support 1 is somewhat radially compressed in its seat within the ear canal 14 which creates a contact force acting radially on the inner ear wall of the outer ear canal 14. This contact force fixes the sensor support 1 securely and detachably in the ear canal 14.

In addition, all those vital parameter sensors whose function requires skin contact with the inner wall of the ear canal, or at least direct optical access, are located on the radially outwardly oriented upper side of the hollow-cylindrically shaped support 1. This concerns in particular the ECG contact surfaces 7 and the light-emitting diode 5 together with the photodetector 6 of the oxygen saturation sensor 5, 6.

In contrast, the body temperature sensor 4 and the heart rate sensor 3 are integrated within the planar substrate 2, in a manner invisible from the outside. Purely for reasons of illustration of the individual sensors, the sensors 3 to 8 are all visible on the surface of the planar substrate 2 in FIG. 1a.

FIG. 2 shows the state of the hollow cylindrical sensor support 1 within the outer ear canal 14 of a person, The radially outer cylinder surface is spring-loaded and nestles against the inner wall of the outer ear canal 14 in a surface-contacting manner. The control unit 10, which is connected to the microelectronics 9 for example via a wire connection, can be miniaturized and fixed inconspicuously behind the auricle 13. Alternatively, it is possible to design the control unit 10 as an external unit, e.g. in the form of a smart phone, using wireless technology to communicate with the microelectronics 9.

The embodiment of a hollow cylindrical sensor support 1 shown in FIG. 3 represents an integral design of hollow cylinder, which is formed from a compressible material, for example a skin-friendly polymer foam. In the same manner as when inserting an ear plug, which is known per se, the support 1 which is equipped with the sensors can also be radially compressed in this manner and inserted into the outer ear canal 14 of a person. The hollow cylindrical support 1 in FIG. 3 is manufactured by a casting process.

In all of the forms of embodiments for a device for the sensory recording of human vital parameters within the outer ear canal 14, the ear canal 14 remains continuously ventilated by virtue of the hollow-cylindrical design of the support 1, so that even permanent use of the device in the ear leads neither to hygienic problems, nor to any hearing restrictions.

The hollow-cylindrical sensor support 1, which is spring-loaded against the inner wall of the outer ear canal, has, as a straight hollow cylinder which has a constant outer diameter over its entire axial hollow-cylindrical extent. Since the natural inner contour of the outer ear canal does not necessarily correspond to the outer surface of a straight cylindrical shape, the hollow cylindrical sensor support may not have uniform surface contact with the outer ear canal. This can result in pulse wave-related deformations of the outer ear canal, which can be measured using the capacitive interdigital structure described above, which are not being fully detected, as a result of only partial contact between the sensor support and the inner wall of the outer ear canal.

In order to avoid this, it is appropriate to form or structure the planar substrate by winding the planar substrate in such a way as to obtain an individual hollow shape that can fit as closely as possible to the inner wall of the outer ear canal.

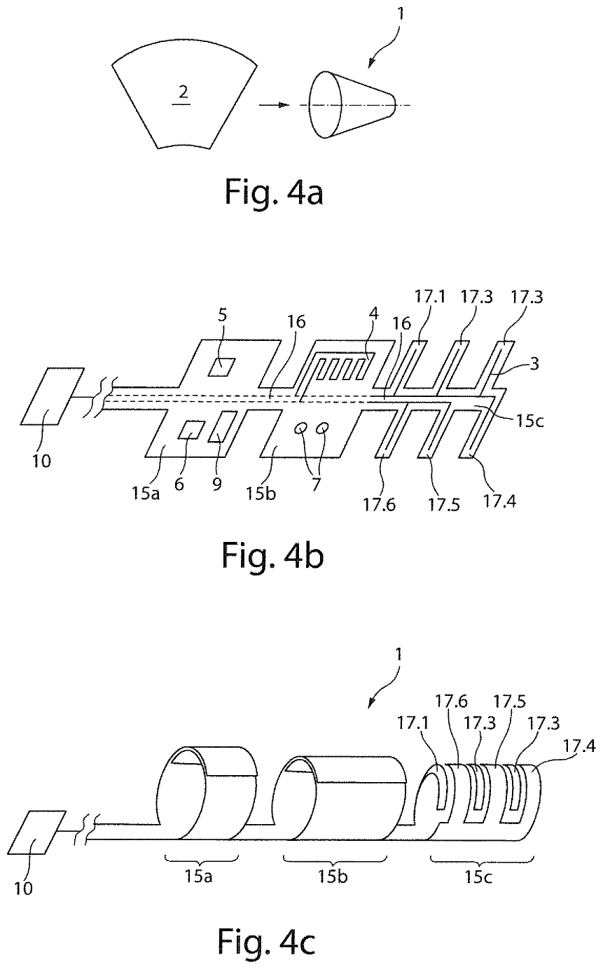

In this context FIG. 4a illustrates a planar substrate 2, in a planar representation on the left-hand side, and in a wound form on the right-hand side. The flat planar substrate 2 has the shape of an annular segment, which in its wound form forms a funnel for insertion into the outer ear canal. The display of the sensors contained in the sensor support 1 is omitted.

FIG. 4b shows another example of embodiment with a structured planar substrate 2, which has surface segments 15a, 15b, and 15c, integrally connected with one another via intermediate web regions 16. The surface segments 15a, 15b, 15c can differ from one another in shape and size. In contrast to the rectangular planar surface segments 15a and 15b, the surface segment 15c is additionally structured so as to have individual radially extendable finger sections 17.1, 17.2, 17.3, 17.4, 17.5, etc. in the wound state, along which interdigital electrodes 3 are inserted for capacitive heart rate measurement. The interrupted finger structure in the surface segment 15c, as compared to a hollow cylinder wall, allows individual contact of the individual finger sections with the individual geometry of the inner wall of the outer ear canal.

The winding process for the transformation of the flat planar substrate into the wound form uses an annealing process, which preserves the hollow cylindrical shape permanently.

Depending on individual circumstances, the intermediate web regions 16 can be suitably selected in terms of width and length. The installation and distribution of the sensors, such as the ECG contact surfaces 7, a light emitting diode 5 together with the photodetector 6 of the oxygen saturation sensor 5, 6, the body temperature sensor 4, as well as the control microelectronics 9, can also be undertaken as required.

The sensor support 1 can, in this case also, be connected wirelessly or in a wired manner to a control unit 10, as already mentioned above.

### LIST OF REFERENCE SYMBOLS

1 Support
2 Planar substrate
3 Heart rate sensor
4 Body temperature sensor
5, 6 Oxygen saturation sensor or CO₂ sensor
7 ECG sensor
8 Other sensors
9 Microelectronics unit
10 Control unit
11 Side edge region
12 Side edge region
13 Auricle
14 Outer ear canal
15a Surface segment
15b Surface segment
15c Surface segment
16 Intermediate web region
17.1 . . . Finger sections
17.5 Finger sections overlap
d1 Hollow cylinder diameter in the state without an external force
d2 Diameter of the outer ear canal

1.-15. (canceled)

16. A device for sensory detection of at least one human vital parameter including a support, which is shaped and dimensioned to be at least partly removable when placed in the human outer ear canal, and onto which at least one sensor for detection of a vital parameter is attached, wherein
the support is a planar substrate, shaped as a hollow cylinder, which provides a continuously open hollow channel having a cylindrical axis in common with the hollow cylinder, which has a hollow cylinder outer wall region which, in state of placement of the support, the hollow cylinder outer wall region at least partly comes into surface contact with the outer ear canal.

17. The device according to claim 16, wherein the planar substrate is converted into a shape of the hollow cylinder by winding around a winding axis.

18. The device according to claim 17, wherein the planar substrate has an inherent shape-retaining stiffness when wound, so that the planar substrate, which is transformed into a hollow cylinder by winding, has two opposing planar substrate sections, which overlap each other loosely as a result of the winding process, without an external mechanical constraint.

19. The device according to claim 18, wherein the planar substrate which is wound into a hollow cylinder without an external mechanical constraint and has a first outer cylinder diameter that is greater than a second outer cylinder diameter, which when wound into the hollow cylinder, exerts a radially outwardly directed force on the outer ear canal.

20. The device according to claim 16, wherein the planar substrate has at least two surface segments which are integrally connected to one another via an intermediate web region so that the surface segments are wound into a hollow cylinder.

21. The device according to claim 20, wherein at least one surface segment in a wound state has individually radially extending finger sections which each mesh with one another in pairs.

22. The device according to claim 16, wherein the planar substrate is integrally formed into a hollow cylinder, the planar substrate is comprises a compressible material and, without an external mechanical constraint has a first outer cylinder diameter that is greater than a second outer cylinder diameter and when the planar substrate is placed on the support when at least partly in the human outer ear canal,
the planar substrate exerts a radially outwardly directed force onto the outer ear canal.

23. The device according to claim 16, wherein the planar substrate comprises at least one layer of polymer material, in which the at least one sensor is integrated or is applied onto the at least one sensor.

24. The device according to claim 16, wherein the at least one sensor is selected from the group of an interdigital electrode arrangement, an electrical resistance sensor, a light sensor system including a light source and light detector, and an acceleration sensor.

25. The device according to claim 16, wherein at least one exposed, metallic surface contact which is connected to an electrical component integrated within the planar substrate is disposed on a hollow cylinder outer wall region facing the outer ear canal.

26. The device according to claim 16, comprising:
a signal and power transmission unit attached to the support which exchanges signals and power with an external terminal device either wirelessly or with wires.

27. The device according to claim 16, wherein the support in the state of placement at least partly in the human outer ear canal has free access to the eardrum through the outer ear canal.

28. The device according to 24, wherein the interdigital electrode arrangement is integrated into the surface segment.

29. The device according to claim 16, wherein at least one actuator is attached to the support.

30. A use of the device according to claim 16, comprising detecting of at least one of heart rate, an ECG signal, body temperature, blood oxygen saturation, CO₂ level, pH value, blood sugar, skin resistance, and blood pressure.
